(19) United States
(12) Patent Application Publication
(10) Pub. No.: US 2023/0302247 A1
(43) Pub. Date: Sep. 28, 2023

(54) Assembly for a patient ventilation system and temperature sensor device to be plugged into a sensor receptacle of a connector of such an assembly

(71) Applicants: New Ventures GmbH, Rehau (DE); RAUMEDIC AG, Münchberg (DE)

(72) Inventors: Ricardo EHRENPFORDT, Zwickau (DE); Constanze RANFELD, Rehau (DE); Lydia GOLBS, Döhlau (DE); Thomas RUHLAND, Bayreuth (DE)

(73) Assignees: New Ventures GmbH, Rehau (DE); RAUMEDIC AG, Münchberg (DE)

(21) Appl. No.: 18/125,205

(22) Filed: Mar. 23, 2023

(30) Foreign Application Priority Data
Mar. 28, 2022 (DE) 10 2022 203 010.9

(51) Int. Cl. A61M 16/08; A61M 16/00

(52) U.S. Cl. CPC A61M 16/0816; A61M 16/0003; A61M 16/0875; A61M 2205/3368

(57) ABSTRACT

An assembly for a patient ventilation system has a respiratory air hose section for guiding ventilation air from a ventilation source to a patient. A connector, connected to the respiratory air hose section, is used to connect the respiratory air hose section to a further component of the patient ventilation system guiding ventilation air. An air guiding connector component of the connector forms an integral component with the respiratory air hose section. The connector has a sensor receptacle for inserting a temperature sensor into the air guiding connector component. The temperature sensor is used to measure a temperature of the ventilation air. A temperature sensor device to be plugged into the sensor receptacle of the assembly has a support body, the temperature sensor protruding beyond the support body, and a handle section to grasp a section of the support body facing away from the temperature sensor.

### CROSS-REFERENCES TO RELATED APPLICATIONS

This application claims the priority of German Patent Application, Serial No. DE 10 2022 203 010.9, filed Mar. 28, 2022, the content of which is incorporated herein by reference in its entirety as if fully set forth herein.

### FIELD OF THE INVENTION

The invention relates to an assembly for a patient ventilation system. Furthermore, the invention relates to a temperature sensor device to be plugged into a sensor receptacle of a connector of such an assembly.

### BACKGROUND OF THE INVENTION

Components for a patient ventilation system are known from CN 213 852 621 U, from WO 2007/051 230 A1, from DE 10 2019 216 489 A1, from U.S. Pat. No. 9,903,371 B2, and from US 2019/0 290 866 A1. Furthermore, a patient ventilation system “VentStar Helix” is known from the market.

### SUMMARY OF THE INVENTION

It is an object of the present invention to provide an assembly for a patient ventilation system, via which a number of air-guiding components of the patient ventilation system to be plugged together is reduced.

This object is achieved according to the invention by an assembly for a patient ventilation system
having a respiratory air hose section for guiding ventilation air from a ventilation source to a patient,
having a connector, connected to the respiratory air hose section, for connecting the respiratory air hose section to a further component of the patient ventilation system guiding the ventilation air, wherein an air guiding connector component of the connector forms an integral component with the respiratory air hose section,
wherein the connector has a sensor receptacle for inserting a temperature sensor into the air guiding connector component to measure a temperature of the ventilation air.

It has been recognized according to the invention that it is possible within an assembly for a patient ventilation system having an integral connector, which is used to connect a respiratory air hose section to a patient interface, to equip this connector at the same time with a sensor receptacle for a temperature sensor. The temperature sensor can then optionally be plugged into the sensor receptacle, if a temperature measurement is required, or the sensor receptacle can be closed using a blank cover if a temperature measurement is not required. A separate sensor connector is avoided using such an assembly, via which the assembly would then be connected to the patient interface. The sensor receptacle can be integrally formed in a housing of the connector. The sensor receptacle can be the result, for example, of an injection moulding production method of the housing of the connector.

The air guiding connector component of the connector on the one hand and the respiratory air hose section on the other are connected in one piece.

The further component of the patient ventilation system guiding the ventilation air, to which the connector of the assembly establishes a connection, can be a patient interface. Alternatively, this further component can be, for example, a humidifier.

Contact pins configured for electrical connection to the temperature sensor or contact pins which are electrically contacted with jacketed wires, which are guided along a hose jacket of the respiratory air hose section, enable a communication of the temperature sensor device with a central controller of the patient ventilation system, in particular via jacketed wires which are guided in the respiratory air hose section. The jacketed wires can be, among other things, heating wires of the respiratory air hose section. The jacketed wires can be guided in a spiral around an air guiding lumen of the respiratory air hose section. Alternatively or additionally to heating wires, a signal wire for transmitting a temperature sensor signal can be embodied as such a jacketed wire. Two or three jacketed wires can be electrically contacted via the contact pins, for example.

A feedthrough of the sensor receptacle for feeding through the temperature sensor into an air guiding lumen of the air guiding connector component enables the use of a temperature sensor which measures the ventilation air temperature directly in the air guiding lumen of the connector. This allows a rapid temperature measurement.

A sensor guiding component for guiding an insertion movement of the temperature sensor into the air guiding lumen of the air guiding connector component can facilitate an insertion of the temperature sensor into the sensor receptacle. The sensor guiding component can be used simultaneously to protect the temperature sensor. The sensor guiding component can have at least one window, so that an air guiding lumen of the air guiding connector component is accessible from the temperature sensor for temperature measurement of the ventilation air guided in the air guiding lumen. The sensor guiding component can contain thermally conductive components for improved connection of the temperature sensor to the air guiding lumen. The sensor guiding component can be an integral component of the sensor receptacle and/or the connector housing.

A sensor receptacle designed for the pluggable insertion of a temperature sensor device, which is part of the temperature sensor, results in a secure mount of the temperature sensor device. The sensor receptacle can have a detent section for locking with a corresponding detent section of a temperature sensor device having the temperature sensor.

A further object of the invention is to provide a temperature sensor device for use with the above-described assembly.

This object is achieved according to the invention by a temperature sensor device to be plugged into a sensor receptacle of a connector of an assembly according to the invention,
having a support body,
having a temperature sensor,
having a handle section for grasping a section of the support body facing away from the temperature sensor.

The temperature sensor device firstly has the advantages which have already been explained above in conjunction with the assembly. The handle section enables secure grasping of the support body of the temperature sensor device and thus secure plugging of the temperature sensor into the sensor receptacle and/or secure removal of the temperature sensor device, possibly to be exchanged for a blank cover. Contact pins of the temperature sensor device, in particular like a socket, enable the electrical contact with the contact pins of the sensor receptacle. The support body and the handle section can thus be integrally connected to one another and can be, for example, the result of a joint injection moulding process.

In particular a temperature value threshold value detection is possible using the temperature sensor. This can be used for medical purposes, but also for other purposes, such as fire protection. An accuracy of the temperature sensor can be better than 0.5 K and can be, for example, 0.2 K, 0.1 K, or also 0.05 K. This accuracy can be ensured in a range between −10° C. and 80° C. A narrower temperature range in which the accuracy is ensured is also possible, for example between 0° C. and 50° C. or between 10° C. and 50° C.

The temperature sensor can protrude beyond the support body. The temperature sensor can be embedded in the support body.

The temperature sensor can have an aerodynamic basic shape, for example a bead shape.

A detent connection formed on the support body for locking with a counter detent section of the sensor receptacle enables a secure mechanical connection of the temperature sensor device to the sensor receptacle. The counter detent section can in turn be formed on the sensor receptacle.

An exemplary embodiment of the invention will be explained in more detail hereinafter on the basis of the drawing.

### BRIEF DESCRIPTION OF THE DRAWING

FIG. 1 shows in perspective an assembly for a patient ventilation system having a respiratory air hose section, a connector connected thereto for connecting the respiratory air hose section to a patient interface, and a sensor receptacle, in which a temperature sensor device is inserted;

FIG. 2 shows a side view, revealing internal details, of the assembly according to FIG. 1, again having inserted temperature sensor device;

FIG. 3 again shows in perspective the assembly in an intermediate stage of its production, wherein a pre-moulded 3D line connection is extrusion coated integrally in each case with contact pins of the sensor receptacle and jacketed wires of the respiratory air hose section to form an inner line carrier housing sleeve of the connector;

FIG. 4 shows partially schematically processing steps during a method for producing the assembly including the production of multiple inner line carrier housing sleeves by extrusion coating and subsequent mechanical separation of 3D line connections initially connected to one another, which were still connected to one another during the extrusion coating step inside a multiple cavity;

FIG. 5 shows further processing steps of the production method up to the production of the finished assembly and a subsequent quality control;

FIG. 6 shows, in an illustration similar to FIG. 3, which shows a core of an ultrasonic welding tool, a further embodiment of the assembly in the intermediate stage of its production, wherein a prepared line connection, embodied as a 2D line connection, is extrusion coated with the contact pins of the sensor receptacle and the jacketed wires of the respiratory air hose section to form the inner line carrier housing sleeve of the connector;

FIG. 7 shows a top view of the completely produced assembly, starting from the intermediate stage according to FIG. 6, wherein the assembly is shown without inserted temperature sensor device.

### DETAILED DESCRIPTION

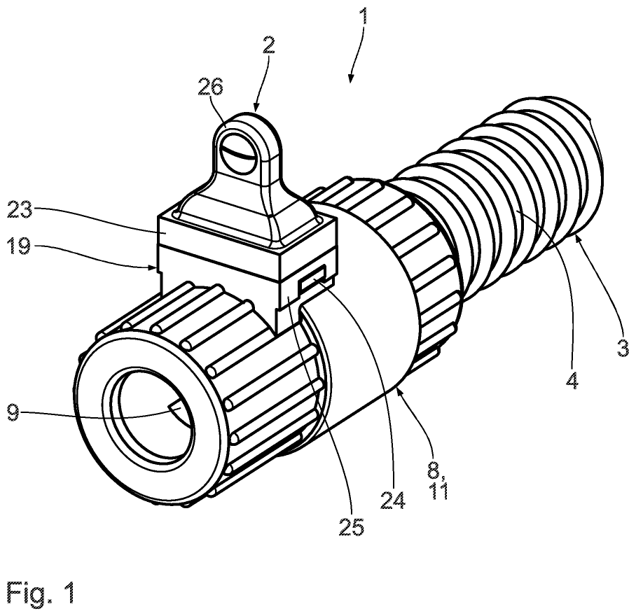

A patient ventilation system, with which an assembly 1 is associated, which is shown in perspective having an inserted temperature sensor device 2 in FIG. 1, is used for ventilating a patient in the area of hospital care, other stationary care, or also household care. In principle, such a patient ventilation system is known from WO 2007/051 230 A1 and DE 10 2019 216 489 A1. A corresponding patient ventilation system is known from the product “VentStar Helix” on the market.

The essential air-guiding components of the ventilation system 1 are made of plastic.

The assembly 1 has a respiratory air hose section 3 for guiding ventilation air from a respiratory air source (not shown) to a patient.

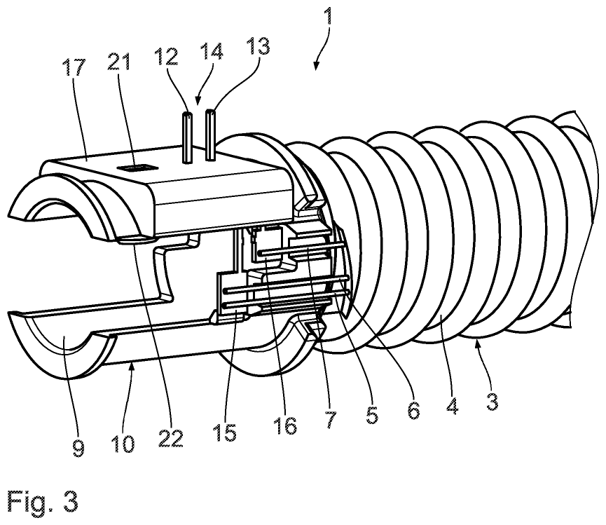

Electrically conductive jacketed wires 5, 6, and 7 (cf. FIG. 3) are guided along a hose jacket 4 of the respiratory air hose section 3. This guide takes place in a spiral around an inner air guiding lumen of the respiratory air hose section 3. The jacketed wires 5 to 7 are extrusion coated for this purpose in a spiral using material of the respiratory air hose section. Two of these jacketed wires, namely the jacketed wires 5 and 6, represent heating wires for the respiratory air hose section 3, which can be supplied with heating current via a supply unit (not shown). The third jacketed wire 7 represents a signal line for the temperature sensor device 2.

A connector 8 of the assembly 1 is connected to the respiratory air hose section 3 by extrusion coating of the respiratory air hose section 3. The connector 8 is used to connect the respiratory air hose section 3 to a patient interface (not shown in the drawing) of the patient ventilation system. This connection is such that an inner air guiding connector component 9 of the connector 8 forms an integral component with the respiratory air hose section 3 at least in sections.

The connector 8 has an inner line carrier housing sleeve 10 and an outer connector housing 11. Those sections of the air guiding connector component 9 which are formed on the respiratory air hose section 3 and thus form an integral component therewith are sections of the outer connector housing 11 of the connector 8. The air guiding connector component 9 and the respiratory air hose section 3 are connected in one piece.

Line components 12, 13 of a three-dimensionally extending 3D line connection of the assembly 1 are moulded into the inner line carrier housing sleeve 10. This 3D line connection will be explained hereinafter.

The line component 12 is electrically connected via a contact pad 15 to the two heating jacketed wires 5, 6 and represents a short-circuit bridge for a heating circuit of the respiratory air hose section 3. The line component 13 is electrically connected via a contact pad 16 to the signal jacketed wire 7. The jacketed wires 5 to 7 are exposed from the spiral-shaped extrusion coating for contacting with the contact pads 15, 16. Facing away from the contact pads 15, 16, the line components 12, 13 end in contact pins which protrude over an insulating connecting plate 17 of the inner line carrier housing sleeve 10. When the temperature sensor device 2 is plugged in, the contact pins of the line components 12, 13 are electrically connected to corresponding contact sockets of the temperature sensor device 2.

The two protruding contact pins of the line components 12, 13 are used for electrically connecting the jacketed wires 5 to 7 to the temperature sensor device 2.

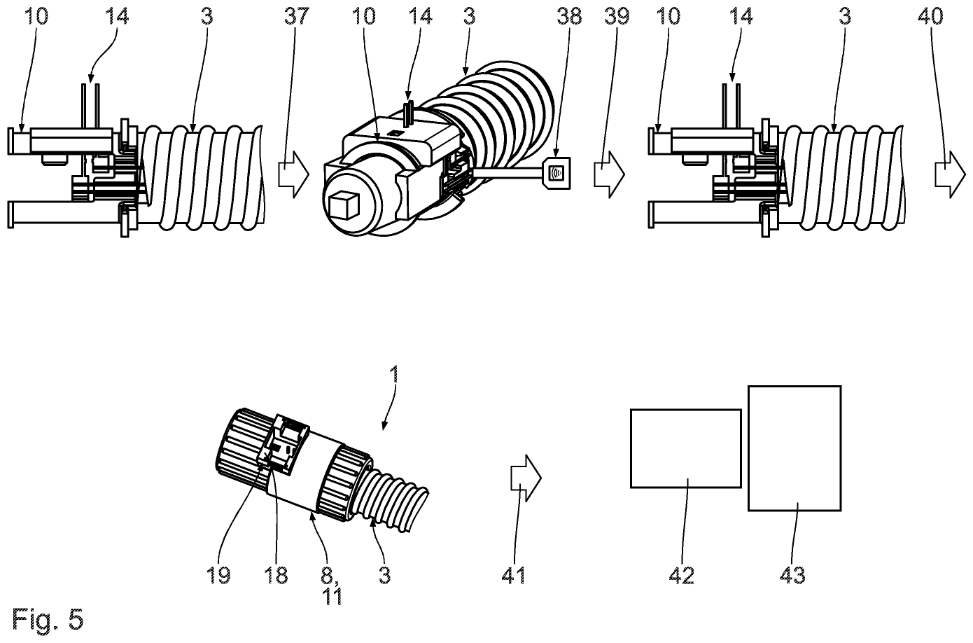

Together with a receptacle body 18 (cf. the next-to-last illustration of FIG. 5), which is part of the outer connector housing 11, the connecting plate 17 represents a sensor receptacle 19 for inserting the temperature sensor device 2 into the connector 8 and in particular for inserting a temperature sensor 20 of the temperature sensor device 2 into the air guiding connector component 9, thus into its air guiding lumen. The sensor receptacle 19 is integrally formed in the outer connector housing 11 of the connector 8. The temperature sensor 20 is used for measuring a temperature of the ventilation air flowing through the air guiding connector component 9.

The temperature sensor device 2 is an example of an electronic component pluggable into the receptacle 19. The sensor receptacle 19 is therefore more generally a component receptacle. Instead of a temperature sensor, a sensor device for acquiring a further parameter, in particular a respiratory air parameter, can also be used in the electronic component.

The sensor device can be a sensor for measuring the ambient humidity or also a chemical or spectroscopic sensor. Another electronic component can also be inserted into the component receptacle 19, for example a monitoring unit, for example, for determining a usage duration or a usage load of the assembly 1.

The sensor receptacle 19 has a feedthrough 21 for feeding through the temperature sensor 20 of the temperature sensor device 2 into the air guiding lumen of the air guiding connector component 9. This feedthrough 21 is embodied as an opening in the connecting plate 17, which opens out into the air guiding lumen of the air guiding connector component 9.

Moreover, the sensor receptacle 19 has a sensor guiding component 22, which is used, inter alia, for guiding an insertion movement of the temperature sensor 20 into the air guiding lumen of the air guiding connector component 9. The sensor guiding component 22 moreover represents a flow and/or contact protection for the temperature sensor 20.

The sensor guiding component 22 can have at least one window, via which the air guiding lumen is accessible from the temperature sensor 20 for temperature measurement of the ventilation air guided in the air guiding lumen. The sensor guiding component 22 can contain thermally conductive components for the thermal connection of the temperature sensor 20 to the air guiding lumen.

The sensor receptacle 19 is designed for the pluggable insertion of the temperature sensor device 2. For this purpose, the temperature sensor device 2 has a support body 23, beyond which the temperature sensor 20 plugged into the sensor receptacle 19 protrudes downward into the air guiding lumen of the air guiding connector component 9. A detent section 24 (cf. FIG. 1) for locking with a counter detent section 25 of the sensor receptacle 19 is formed on the support body 23. The counter detent section 25 is in turn a component formed on the sensor receptacle 19.

Contact pins of the temperature sensor device for connection to the contact pins of the line components 12, 13 are provided in the support body 23.

A handle section 26 of the temperature sensor device 2 for grasping a section of the support body 23 facing away from the temperature sensor 20 is also formed on the support body 23.

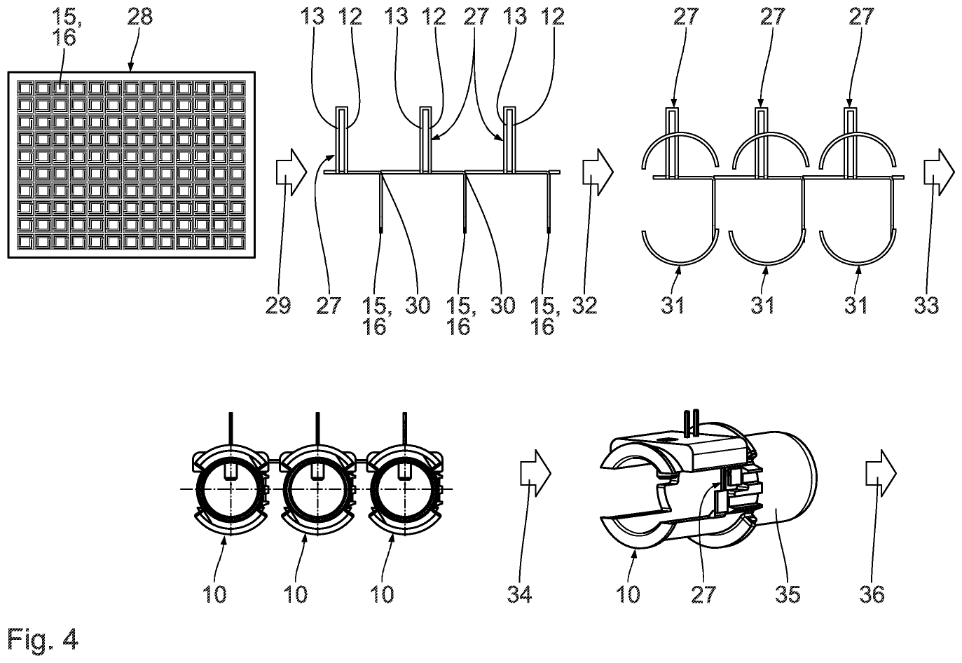

A method for producing the assembly 1 is explained in more detail hereinafter on the basis of FIGS. 4 and 5.

To produce the assembly 1, first a three-dimensionally extending line connection 27 is pre-moulded starting from a 2D conductor structure 28 designed as a lead frame. The line components of the 2D line structure 28 can have cross sections which are smaller than 0.5 mm and can be in the range between 50 μm and 200 μm.

The respective 3D line connection 27 extends between the respective contact pads 15, 16 and the contact pins of the line components 12, 13. A plurality of contact pads 15 or 16, respectively, are connected to one another in the 2D conductor structure 28 via corresponding conductor tracks, which are bent over in the context of a pre-moulding step 29 in relation to one another, in particular by 90° to the 3D line connection 27.

The result of the preform step 29 is a plurality of 3D line connections 27, in the present example three such 3D line connections 27, which are assigned to a corresponding plurality of inner line carrier housing sleeves 10 of a respective connector 8 to be formed on later. These multiple 3D line connections 27 are initially still connected to one another mechanically via intended breakpoints 30 after the pre-moulding step 29.

After the pre-moulding 29 of the 3D line connections 27, the 3D line connections 27 which are still mechanically connected to one another are inserted into an injection mould in the form of an injection moulding multiple cavity 31, which takes place in an insertion step 32. Subsequently thereto, in an extrusion coating step 33, extrusion coating of the 3D line connections 27 takes place to form the inner line carrier housing sleeves 10 of the respective connector 8. During the extrusion coating step 33, the plurality of the 3D line connections 27 still mechanically connected to one another are extrusion coated in the injection moulding multiple cavity 31 to form the corresponding plurality of inner line carrier housing sleeves 10.

After the extrusion coating step 33, in a separating step 34, a mechanical separation of the 3D line connections 27 connected to one another at the intended breakpoints 30 takes place and thus an isolation of the inner line carrier housing sleeves 10.

Subsequently thereto, a connecting sleeve 35 of the isolated inner line carrier housing sleeves 10 is plugged into an end section of the respiratory air hose section 3 facing toward it in a plugging-in step 36.

FIG. 5 shows on the top left an inner line carrier housing sleeve 10 correspondingly plugged into the respiratory air hose section 3. In the course of the plugging-in step 36, the contact pads 15, 16 of the line components 12, 13 are positioned and aligned toward the exposed end sections of the jacketed wires 5 to 7.

In a subsequent connecting step 37, the line components 12, 13 of the line connection 14 or 27 are electrically contacted with the end sections of the jacketed wires 5 to 7. This takes place by means of ultrasonic welding using a correspondent ultrasonic welding device 38.

Subsequently, in an insertion step 39, the prefinished raw assembly having the inner line carrier housing sleeves 10 and the respiratory air hose section 3 mechanically and electrically connected thereto is inserted into a further injection mould. In a subsequent further extrusion coating step 40, the inner line carrier housing sleeves 10 and an end area of the respiratory air hose section 3 adjoining thereon are extrusion coated to form the outer connector housing 11. The sensor receptacle 19 is also formed in the outer connector housing in this case.

The electronic component which can be accommodated in the component or sensor receptacle 19 can in general be a sensor device, thus, for example, the temperature sensor device 2.

After the further extrusion coating step 40, an optical quality control 42 and an electrical quality control 43 take place in a control step 41, wherein the assembly 1 can be subjected to a visual check and a check by corresponding optical and/or electrical/electronic measuring units.

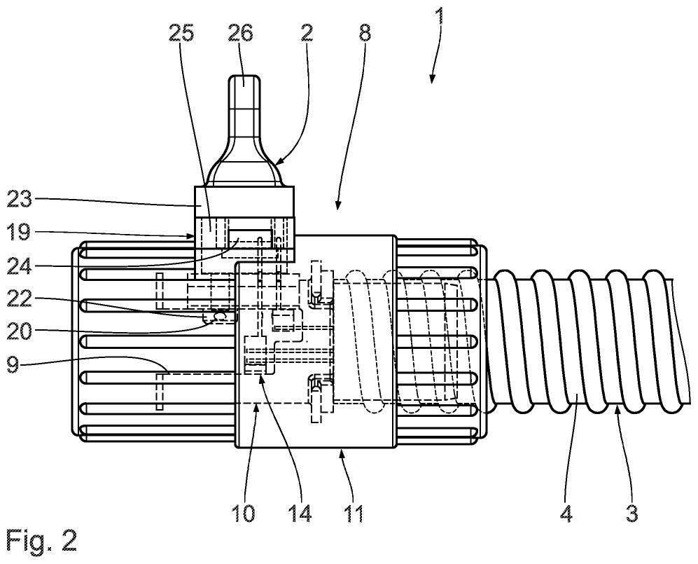

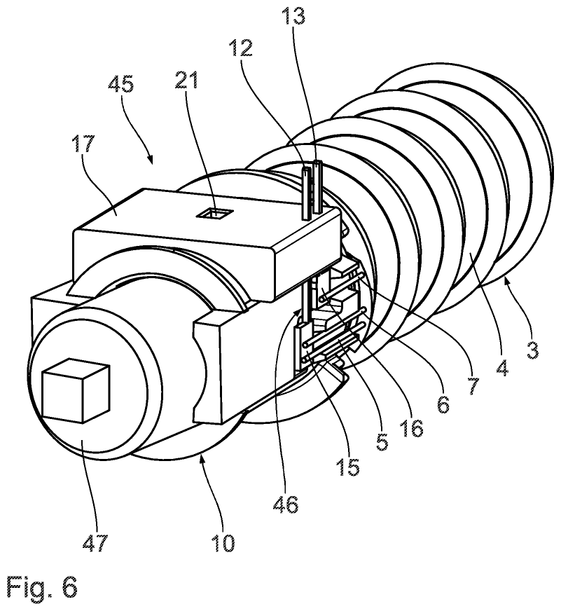

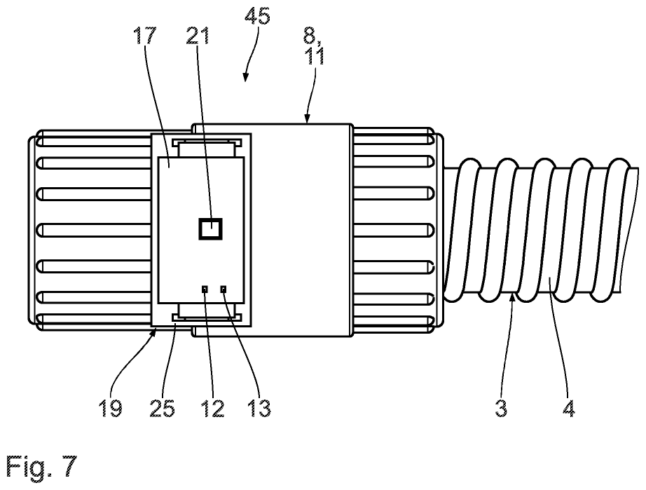

A further embodiment 45 of the assembly for the patient ventilation system will be described hereinafter on the basis of FIGS. 6 and 7, which can be used instead of the assembly which was described above on the basis of FIGS. 1 to 5. Components and functions which correspond to those which were already explained above with reference to FIGS. 1 to 5 bear the same reference signs and are not discussed in detail once again.

In the assembly 45, a line connection 46, which otherwise corresponds to the 3D line connection 27 of the embodiment according to FIGS. 1 to 5, is embodied as two-dimensional. The contact pins 12, 13 of this line connection and the contact pads 15, 16 lie in a common arrangement plane of the 2D line connection 46.

FIG. 6 moreover shows a core 47 of an ultrasonic welding tool for the ultrasonic welding of the line components 12, 13 of the line connection 14 or 27, respectively, to the end sections of the jacketed wires 5 to 7 of the assembly 45.

The production method of the assembly 45 corresponds in principle to that which was already explained above on the basis of FIGS. 4 and 5.

Instead of pre-moulding of a 3D line connection, in the production of the assembly 45, initially a provision of the 2D line connection 46 starting from the 2D conductor structure 28 embodied as a lead frame takes place. This provision can be carried out exclusively by separating the associated line components from surrounding support or line components of the lead frame 28.

The result of this provision of the 2D line connections 46 is a plurality of such 2D line connections 46, for example three such 2D line connections 46, which are assigned to a corresponding plurality of the inner line carrier housing sleeves 10, to be formed on later, of the respective connector 8 of the assembly 45. These multiple 2D line connections 46 are initially still mechanically connected to one another via intended breakpoints after the provision step, as was already explained above on the basis of the 3D line connections 27 of the embodiment according to FIGS. 1 to 4.

In the production of the assembly 45, the insertion 32, the extrusion coating, the isolation 34, the plugging in of the connection sleeves 35 including the positioning and alignment of the contact pads 15, 16 in relation to the exposed end sections of the jacketed wires 5 to 7, the electrical connection 37, the insertion 39, the further extrusion coating 40, and the control 41 then take place corresponding to that which was already explained above in conjunction with the production of the assembly 1 according to FIGS. 1 to 5.

What is claimed is:

1. An assembly (1; 45) for a patient ventilation system
having a respiratory air hose section (3) for guiding ventilation air from a ventilation source to a patient,
having a connector (8), connected to the respiratory air hose section (3), for connecting the respiratory air hose section (3) to a further component of the patient ventilation system guiding the ventilation air, wherein an air guiding connector component (9) of the connector (8) forms an integral component with the respiratory air hose section (3),
wherein the connector (8) has a sensor receptacle (19) for inserting a temperature sensor (20) into the air guiding connector component (9) to measure a temperature of the ventilation air.

2. The assembly according to claim 1, wherein the sensor receptacle (19) has at least two contact pins (12, 13) for electrical connection to the temperature sensor (20).

3. The assembly according to claim 2, wherein the at least two contact pins (12, 13) are electrically contacted with jacketed wires (5 to 7), which are guided along a hose jacket (4) of the respiratory air hose section (3).

4. The assembly according to claim 1, wherein the sensor receptacle (19) has a feedthrough (21) for feeding through the temperature sensor (20) into an air guiding lumen of the air guiding connector component (9).

5. The assembly according to claim 4, wherein the sensor receptacle (19) has a sensor guiding component (22) for guiding an insertion movement of the temperature sensor (20) into the air guiding lumen of the air guiding connector component (9).

6. The assembly according to claim 1, wherein the sensor receptacle (19) is designed for pluggable insertion of a temperature sensor device (2), which is part of the temperature sensor (20).

7. A temperature sensor device (2) to be plugged into a sensor receptacle (19) of a connector (8) of an assembly (1) for a patient ventilation system,
having a support body (23),
having a temperature sensor (20),
having a handle section (26) for grasping a section of the support body (23) facing away from the temperature sensor (20).

8. The temperature sensor device according to claim 7, comprising a detent section (24) formed on the support body (23) for locking with a counter detent section (25) of the sensor receptacle (19).
